(19) United States
(12) Patent Application Publication
(10) Pub. No.: US 2023/0293573 A1
(43) Pub. Date: Sep. 21, 2023

(54) OHPP-FORMULATED NICLOSAMIDE TO TREAT SARS-COV-2, OTHER VIRAL DISEASES, AND CANCERS

(71) Applicant: Purdue Research Foundation, West Lafayette, IN (US)

(72) Inventors: Yuan Yao, West Lafayette, IN (US); Jingmin Joanne Zhang, West Lafayette, IN (US)

(73) Assignee: Purdue Research Foundation, West Lafayette, IN (US)

(21) Appl. No.: 18/017,396

(22) PCT Filed: Jul. 23, 2021
(86) PCT No.: PCT/US2021/042868

§ 371 Date: Jan. 21, 2023

Related U.S. Application Data
(60) Provisional application No. 63/181,612, filed on Apr. 29, 2021.
(60) Provisional application No. 63/055,415, filed on Jul. 23, 2020.

(51) Int. Cl. A61K 31/716; A61K 31/167; A61K 45/06; A61P 31/14

(52) U.S. Cl. CPC A61K 31/716; A61K 31/167; A61K 45/06; A61P 31/14

(57) ABSTRACT

The present invention generally relates to a pharmaceutical formulation that substantially increases the bioavailability of niclosamide or a pharmaceutically acceptable salt thereof for a variety of routes of delivery for therapeutic purposes. The newly enhanced formulation also substantially expands the potential therapeutic applications of niclosamide. Pharmaceutical compositions and methods of uses thereof are within the scope of this disclosure.

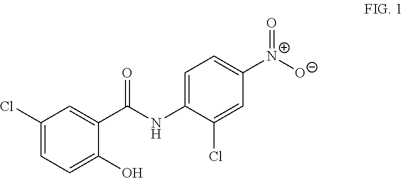 [SMILES: O=C(Nc1ccc([N+](=O)[O-])cc1Cl)c1cc(Cl)ccc1O]

### CROSS-REFERENCE TO RELATED APPLICATIONS

The present U.S. patent application is related to and claims the priority benefit of U.S. Provisional Patent Application Ser. No. 63/055,415, filed Jul. 23, 2020, and U.S. Provisional Patent Application Ser. No. 63/181,612, filed Apr. 29, 2021, the contents of which are hereby incorporated by reference into the present disclosure in their entirety.

### TECHNICAL FIELD

The present invention generally relates to a pharmaceutical formulation that substantially increases the bioavailability of niclosamide or a pharmaceutically acceptable salt thereof for a variety of routes of delivery. The newly enhanced formulation also substantially expands the therapeutic applications of niclosamide. Pharmaceutical compositions and methods of uses thereof are within the scope of this disclosure.

### BACKGROUND

This section introduces aspects that may help facilitate a better understanding of the disclosure. Accordingly, these statements are to be read in this light and are not to be understood as admissions about what is or is not prior art.

Niclosamide is a commercially available medication used in the treatment of tapeworm infestations. However, due to poor solubility, the dosage is high, usually 1000 to 2000 mg. More recently, niclosamide has been found to have potential benefits as an anti-viral treatment. For example, among all potential Covid-19 therapeutic drugs, niclosamide holds great potential due to its demonstrated high (in vitro) efficacy to inhibit SARS-CoV-2 virus and its status as an FDA-approved drug, though currently discontinued. However, niclosamide is poorly water-soluble, which prevents its effective and consistent absorption in the human body. Thus, there is a need for a formulation of niclosamide of higher solubility that is currently available.

Phytoption's OHPP (octenylsuccinate hydroxypropyl phytoglycogen) increases the solubility of niclosamide by a factor of more than >5,000 times, which is at least 50 times greater than leading commercial solubilizers (HPMC-AS, SOLUPLUS®). Importantly, OHPP-formulated niclosamide can be effectively released to cells to provide biological efficacies.

Higher and more consistent bioavailability is an advantage of OHPP-formulated niclosamide. Niclosamide is a BCS class II drug (high permeability, low solubility), and thus the most effective approach to improve niclosamide absorption is to increase its solubility. Thus, the OHPP approach is able to substantially improve the bioavailability of niclosamide over current formulation approaches.

Two independent studies of Jeon et al. (2020) and Gassen et al. (2020) have shown the strong efficacy of niclosamide to inhibit SARS-CoV-2, reporting IC₅₀ values of 0.28 μM (92 ng/mL) and 0.17 μM (56 ng/mL), respectively. A clinical study by Schweizer et al. (2018) highlighted two problems when given oral doses of non-solubilized niclosamide: (1) large variance of plasma concentration that prevented the drug to be effective in patients, and (2) side effects mostly related to the GI tract. Both problems are believed to result from the low solubility of the niclosamide used in the trial. In contrast, a preclinical evaluation of nano-formulated niclosamide (Lin et al, 2016) showed that soluble niclosamide not only increased the oral bioavailability, but also suppressed tumor growth without obvious toxicity at an oral dose of 100 mg/kg with mice; however, their formulation was highly complicated and not feasible for mass manufacturing or use.

There are also other publications showing that niclosamide is effective on other viruses including Influenza, Dengue, Zika, SARS-CoV, Chikungunya virus, and etc. For the same reason of poor solubility, the research on niclosamide as a potential treatment toward those viral infections has not been pursued due to the significant bioavailability challenges.

Based on these data, OHPP-formulated niclosamide may achieve desirable plasma concentration of niclosamide with substantially enhanced consistency and reduced drug dose to alleviate side effects. These factors may greatly improve the therapeutic window of niclosamide-based medicaments for a broad range of Covid-19 patients of various ages, genders, and health conditions, and treating other viral diseases.

By Apr. 21, 2021, the Covid-19 pandemic have caused over 143 million infections and over 3 million deaths globally (Worldometers). Among the therapeutics clinically evaluated, remdesivir has shown promise but with limited efficacy (Beigel et al., 2020) and the use of hydroxychloroquine has caused concerns (Watson et al., 2020). With the reopening of the world economy, there is an enormous urgency for developing alternative therapeutics with much greater efficacy. Among all potential drug candidates, niclosamide stands out due to its FDA status as having been approved decades ago and its high in vitro potency against SARS-CoV-2.

### SUMMARY OF THE DISCLOSURE

In some illustrative embodiments of the disclosure, compositions containing or comprising octenylsuccinate hydroxypropyl phytoglycogen (OHPP) and niclosamide are provided.

In other illustrative embodiments of the disclosure, methods for treating virus infections comprising administering to patients in need thereof an effective amount of compositions comprising OHPP and niclosamide are provided.

In yet additional illustrative embodiments of the disclosure, methods for preventing virus infections comprising administering to patients in need thereof an effective amount of compositions comprising OHPP and niclosamide are provided.

In still additional aspects of the disclosure, methods for treating prostate cancer, breast cancer, lung cancer, ovarian cancer, or other types of cancer comprising administering to patients in need thereof an effective amount of compositions comprising OHPP and niclosamide are provided.

In further aspects of the disclosure, compositions comprising OHPP and niclosamide and one or more additional active agents are provided.

In yet further aspects of the disclosure, methods for treating virus infections comprising administering to patients in need thereof an effective amount of a composition comprising OHPP and niclosamide and further administering an effective amount of one or more additional active agents are provided.

### BRIEF DESCRIPTION OF DRAWINGS

FIG. 1 depicts the chemical structure of niclosamide.

FIG. 2 shows IC₅₀ of niclosamide as compared with that of chloroquine and remdesivir, copied from Jeon et al. (2020).

FIG. 3 demonstrates concentration-dependent inhibition of SARS-CoV-2 growth by spermidine, MK-2206, and niclosamide, copied from Gassen et al. (2020).

FIG. 4 depicts the biopharmaceutical classification system (BCS), wherein niclosamide is a Class II drug.

FIG. 5 depicts the schematic of niclosamide incorporated with OHPP nano-particulate, adapted from Xie and Yao et al. (2018b).

FIG. 6 shows the comparison of the dispersion of niclosamide alone (left) and OHPP-formulated niclosamide (right), each solution containing 2.5 mg/mL of niclosamide in a phosphate buffer (Xie and Yao (2018)).

FIG. 7 demonstrates the dose-efficacy relationship of niclosamide solubilized by DMSO and OHPP, the dotted lines showing the estimation of the IC50 against prostate cancer cell PC-3.

FIG. 8 shows the niclosamide concentration in plasma vs. time and “area under the curve (AUC)” for mice gavaged with insoluble niclosamide (20 or 80 mg/kg) or OHPP-formulated niclosamide (20 mg/kg).

FIG. 9 shows the lung virus titers of hACE2 transgenic mice at 5 days post infection.

FIG. 10 depicts the potential pathways (the first column) that this current disclosure may target for the treatment of relevant diseases (the top row).

### DETAILED DESCRIPTION

While the concepts of the present disclosure are illustrated and described in detail in the description herein, results in the description are to be considered as exemplary and not restrictive in character; it being understood that only the illustrative embodiments are shown and described and that all changes and modifications that come within the spirit of the disclosure are desired to be protected.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided and may be administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

The present invention generally relates to compounds useful for the treatment of various types of infection diseases and cancers. Pharmaceutical compositions and methods for treating those diseases are within the scope of this invention.

In some illustrative embodiments, the present disclosure relates to a composition comprising a highly branched glucan and niclosamide, or a pharmaceutically acceptable salt, hydrate, and solvate thereof.

In some illustrative embodiments, the present disclosure relates to a composition comprising a highly branched glucan and niclosamide, or a pharmaceutically acceptable salt, hydrate, and solvate thereof, wherein the oral bioavailability of niclosamide of said composition is at least 2 times higher than that of niclosamide alone as a pharmaceutical composition.

In some illustrative embodiments, the present disclosure relates to a composition comprising a highly branched glucan and niclosamide, or a pharmaceutically acceptable salt, hydrate, and solvate thereof, wherein said composition further comprises one or more pharmaceutically acceptable excipients or diluents.

In some illustrative embodiments, the present disclosure relates to a composition comprising a highly branched glucan and niclosamide, or a pharmaceutically acceptable salt, hydrate, and solvate thereof, wherein said composition is formulated for oral delivery.

In some illustrative embodiments, the present disclosure relates to a composition comprising a highly branched glucan and niclosamide, or a pharmaceutically acceptable salt, hydrate, and solvate thereof, wherein said composition further comprises one or more absorption enhancers for further improved oral bioavailability of niclosamide.

In some illustrative embodiments, the present disclosure relates to a composition comprising a highly branched glucan and niclosamide, or a pharmaceutically acceptable salt, hydrate, and solvate thereof, wherein said composition is a pill, tablet, capsule, liquid, gel or powder.

In some illustrative embodiments, the present disclosure relates to a composition comprising a highly branched glucan and niclosamide, or a pharmaceutically acceptable salt, hydrate, and solvate thereof, wherein said composition is for inhalation, intranasal, intravenous, intramuscular, subcutaneous, or intradermal delivery.

In some illustrative embodiments, the present disclosure relates to a composition comprising a highly branched glucan and niclosamide, or a pharmaceutically acceptable salt, hydrate, and solvate thereof, wherein said highly branched glucan is octenylsuccinate hydroxypropyl phytoglycogen (OHPP).

In some illustrative embodiments, the present disclosure relates to a method for treating virus infections comprising administering to a patient in need thereof an effective amount of a composition as disclosed herein.

In some illustrative embodiments, the present disclosure relates to a method for treating virus infections comprising administering to a patient in need thereof an effective amount of a composition as disclosed herein, wherein the virus infection is caused by a virus selected from group consisting of SARS-CoV-2, SARS-CoV, MERS-CoV, Influenza A, Dengue virus, Zika virus, Ebola virus, Hepatitis C virus, Japanese encephalitis virus, Human rhinoviruses, Chikungunya virus, Human adenovirus, Epstein-Barr virus, other Influenza, HIV, Polio virus, other viruses, or a combination thereof.

In some other illustrative embodiments, the present disclosure relates to a method for treating a cancer comprising administering to a patient in need thereof an effective amount of a composition as disclosed herein.

In some other illustrative embodiments, the present disclosure relates to a method for treating a cancer comprising administering to a patient in need thereof an effective amount of a composition as disclosed herein, wherein the cancer is prostate cancer, adrenocortical carcinoma, breast cancer, colon cancer, glioma, head and neck cancer, leukemia, lung cancer, osteosarcoma, ovarian cancer, prostate cancer, renal cell carcinoma, or ovarian cancer.

In some other illustrative embodiments, the present disclosure relates to a method for treating a cancer comprising administering to a patient in need thereof an effective amount of a composition as disclosed herein, wherein said patient is an animal or a human being.

In some illustrative embodiments, the present disclosure relates to a method for treating virus infections comprising administering to a patient in need thereof an effective amount of a composition as disclosed herein, wherein the patient is a human being or an animal.

In some illustrative embodiments, the present disclosure relates to a method for treating virus infections comprising administering to a patient in need thereof an effective amount of a composition as disclosed herein, wherein the virus is SARS-CoV-2.

In some illustrative embodiments, the present disclosure relates to a method for treating virus infections comprising administering to a patient in need thereof an effective amount of a composition as disclosed herein, wherein the viral infection is caused by a virus selected from group consisting of SARS-CoV-2, SARS-CoV, MERS-CoV, Influenza A, Dengue virus, Zika virus, Ebola virus, Hepatitis C virus, Japanese encephalitis virus, Human rhinoviruses, Chikungunya virus, Human adenovirus, Epstein-Barr virus, other Influenza, HIV, Polio virus, other viruses, and their combinations.

In some other illustrative embodiments, the present disclosure relates to a pharmaceutical composition comprising the composition as disclosed herein together with one or more additional pharmaceutically active agents.

In some other illustrative embodiments, the present disclosure relates to a pharmaceutical composition comprising the composition as disclosed herein together with one or more additional pharmaceutically active agents, wherein said one or more additional pharmaceutically active agents are an antiviral agent, antibacterial agent, antifungal agent, and/or an anti-inflammation agent.

In some other illustrative embodiments, the present disclosure relates to a method for treating virus infection comprising administering to a patient in need thereof an effective amount of a composition as disclosed herein together with an effective amount of one or more additional pharmaceutically active agents.

In some other illustrative embodiments, the present disclosure relates to a method for treating virus infection comprising administering to a patient in need thereof an effective amount of a composition as disclosed herein together with an effective amount of one or more additional pharmaceutically active agents, wherein the one or more additional active agents are an antiviral agent, antibacterial agent, antifungal agent, and/or an anti-inflammation agent.

In some other illustrative embodiments, the present disclosure relates to a method for treating a cancer comprising administering to a patient in need thereof an effective amount of a composition as disclosed herein together with an effective amount of one or more additional pharmaceutically active agents.

In some other illustrative embodiments, the present disclosure relates to a method for treating a cancer comprising administering to a patient in need thereof an effective amount of a composition as disclosed herein together with an effective amount of one or more additional pharmaceutically active agents, wherein the one or more additional active agents are an anticancer agent.

In some other illustrative embodiments, the present disclosure relates to a method for treating an infection disease comprising administering to a patient in need thereof an effective amount of a composition as disclosed herein together with an effective amount of one or more additional pharmaceutically active agents.

In some other illustrative embodiments, the present disclosure relates to a method for treating an infection disease comprising administering to a patient in need thereof an effective amount of a composition as disclosed herein.

In some other illustrative embodiments, the present disclosure relates to a method for treating an infection disease comprising administering to a patient in need thereof an effective amount of a composition as disclosed herein together with an effective amount of one or more additional pharmaceutically active agents.

In some other illustrative embodiments, the present disclosure relates to a method for treating an infection disease comprising administering to a patient in need thereof an effective amount of a composition as disclosed herein, wherein the infection is a bacterial infection caused by tuberculosis, anthrax, pseudomonas aeruginosa, or staphylococcus aureus, a type 2 diabetes mellitus, nonalcoholic fatty liver disease, artery constriction, endometriosis, neuropathic pain, rheumatoid arthritis, sclerodermatous graft-versus-host disease, or a systemic sclerosis.

In some other illustrative embodiments, the present disclosure relates to a method for treating a disease comprising administering to a patient in need thereof an effective amount of a composition as disclosed herein, wherein said disease is a bacterial infection caused by tuberculosis, anthrax, pseudomonas aeruginosa, or staphylococcus aureus, a type 2 diabetes mellitus, nonalcoholic fatty liver disease, artery constriction, endometriosis, neuropathic pain, rheumatoid arthritis, sclerodermatous graft-versus-host disease, or a systemic sclerosis.

In some other illustrative embodiments, the present disclosure relates to a method for targeting one of more signaling pathways & biological processes including but not limited to uncoupling of oxidative phosphorylation, Wnt, mTORC1, STAT3, NF-□B, Notch, AKT/ERK/Src, AR-V7, C-Fos, C-Jun, E2F1, c-Myc, mGluRs, Metabolic pathways, ROS, Mitochondria, pH, comprising administering to a patient in need thereof an effective amount of a composition as disclosed herein.

In some illustrative embodiments, the present disclosure relates to a composition comprising a highly branched glucan and niclosamide, or a pharmaceutically acceptable salt, hydrate, and solvate thereof, wherein said highly branched glucan is octenylsuccinate hydroxypropyl phytoglycogen (OHPP), and wherein OHPP and niclosamide are incorporated using a method selected from spray drying, spray chilling, extrusion, hot-melt extrusion, homogenization, milling such as jet milling, cryo-milling, ball milling, and colloid milling, vacuum drying, drum drying, film-forming, surface coating, printing, 3D printing, multi-layer forming, high-intensity blending, co-crystallization, personalized manufacturing of dosage forms, and the combination of different procedures.

### Niclosamide is a Potential Covid-19 Therapeutic

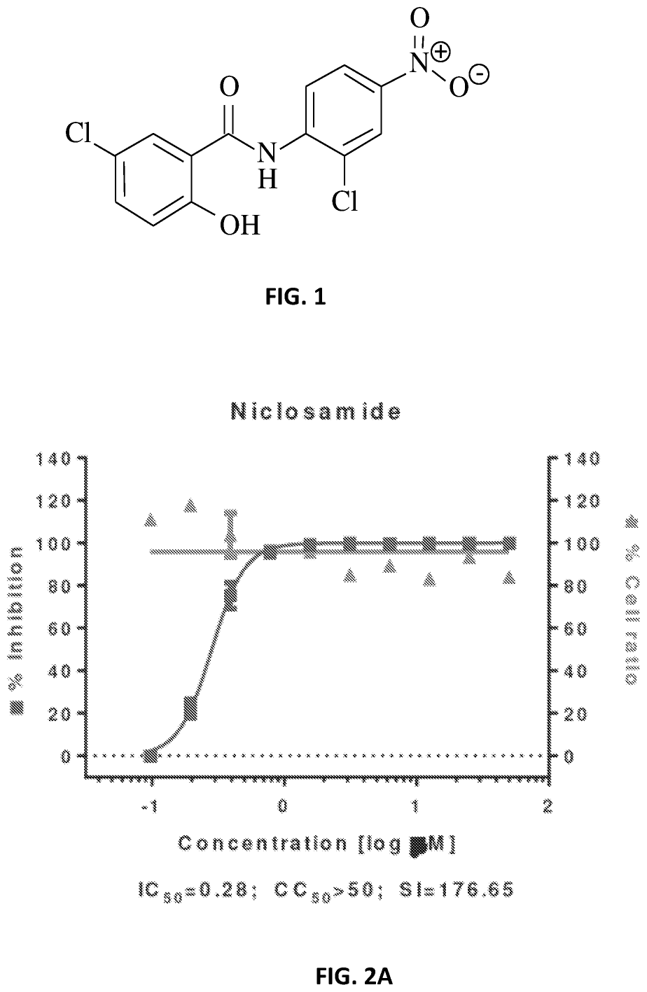

On WHO's Model List of Essential Medicines, Niclosamide (2′,5-Dichloro-4′-nitrosalicylanilide, FIG. 1) is an anthelmintic drug used to treat tapeworm infestations (WHO, 2019). Studies have also shown that niclosamide is a promising anticancer agent against various human cancers (Li et al., 2014) and a potential antiviral agent against a broad variety of viral infections such as SARS-CoV, MERS-CoV, ZIKV, JEV, EBV, EBOV, HRV, CHIKV, HAdV, HCV, Dengue, and human adenovirus (Xu et al., 2020).

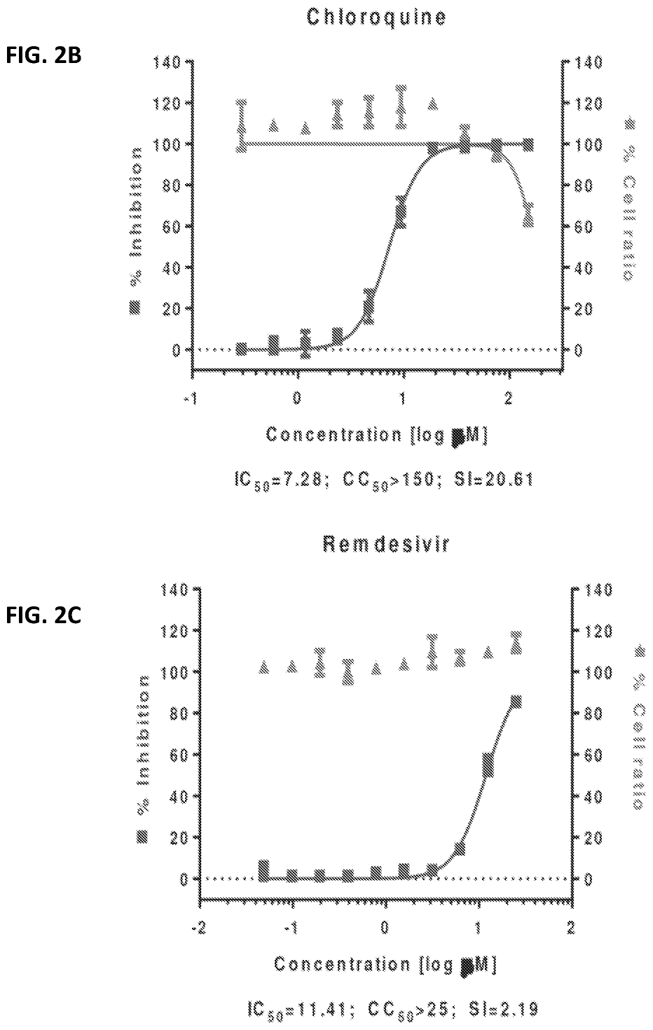

In the past several months, a number of reports have been published to associate niclosamide with antiviral efficacy against SARS-CoV-2. Among those, two studies have independently shown the high efficacy of niclosamide against SARS-CoV-2 with in vitro setting. FIG. 2 showed the result by Jeon et al. (2020) in using Vero cells to compare the anti-SARS-CoV-2 efficacy of various compounds. The half maximal inhibitory concentration (IC₅₀) of niclosamide was 0.28 μM, much lower than that of chloroquine (7.28 μM) and remdesivir (11.41 μM). Meanwhile the selectivity index (SI), which is the ratio of CC₅₀ (50% cytotoxic concentration) to IC₅₀, was 176.65 for niclosamide, much greater than that of chloroquine (20.61) and remdesivir (2.19), suggesting a potentially high safety level for niclosamide as a Covid-19 therapeutic.

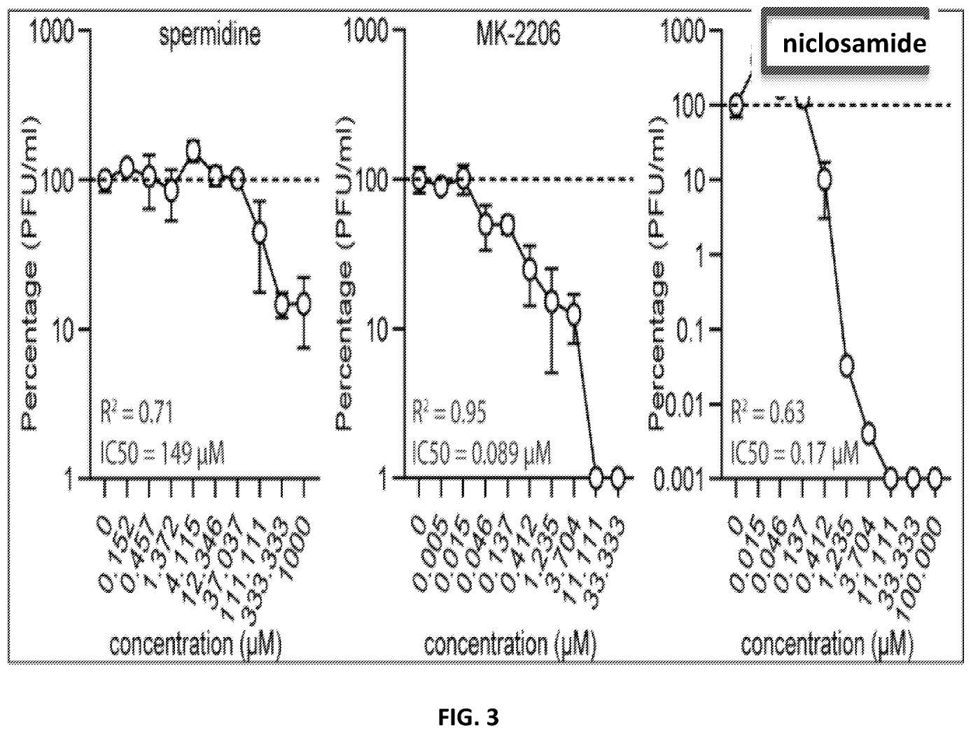

FIG. 3 shows the result by Gassen et al. (2020) in comparing the anti-SARS-CoV-2 efficacy of niclosamide with that of the other two compound (spermidine and MK-2206). The IC₅₀ of niclosamide was 0.17 μM, similar to the value (0.28 μM) reported by Jeon et al. (2020). Therefore, the results from Jeon et al. (2020) and Gassen et al. (2020) corroborated well with each other.

In general, niclosamide is a potential anticancer and antiviral agent against a broad variety of viruses including coronaviruses (e.g. SARS, MERS), Influenza, Dengue, etc. Particularly, niclosamide has shown strong in vitro antiviral efficacy against SARS-CoV-2. The IC₅₀ of 0.28 μM (92 ng/mL) and 0.17 μM (56 ng/mL) can be used as preliminary goals of plasma concentration for effective therapeutic effect. IC₉₀ can also be used to set goals for plasma concentration. Based on FIG. 2, the IC₉₀ of niclosamide was about 0.7 μM (229 ng/mL). These numbers (i.e., 56, 92, and 229 ng/mL) are used in our PK study as benchmarks to evaluate the potential of niclosamide formulations in reaching therapeutic levels in plasma.

### Insoluble Niclosamide Causes Large Plasma Variance and Local Side Effects

Due to its potential efficacy against SARS-CoV, Chang et al. (2006) studied the pharmacokinetics of niclosamide using rats as model animals. In this study, niclosamide was dissolved in a solvent mixture (DMSO/cremophor EL/water volume ratio of 3/15/82, be noted that this solvent cannot be used for humans since it contains DMSO). A single oral dose of 5 mg/kg was used, and niclosamide showed the highest plasma concentration (Cₘₐₓ) of 354±152 ng/mL. This result suggests that soluble niclosamide was able to provide a high plasma concentration. However, the variance was rather large possibly due to rapid crystallization of niclosamide as affected by different GI tract conditions of individual animals. It would be difficult for crystallized niclosamide to effectively penetrate the intestinal epithelial layer.

A phase I clinical study by Schweizer et al. (2018) provided valuable insights regarding the impact of niclosamide solubility on its pharmacokinetics and toxicity. Due to its poor solubility and oral bioavailability, niclosamide was used in large doses, i.e. 500 or 1000 mg, three-times daily (TID) and long times (4 weeks). The data showed that: (1) the maximal plasma concentration reached considerably high level (182 ng/mL) with large variances (35.7-182 ng/mL for 500 mg TID, and 149-182 mg/mL for 1000 mg TID with side effects), (2) the side effects were mostly associated with the GI tract, probably due to a large amount of niclosamide locally retained. Evidently, the variance of plasma concentration and side effects need to be significantly reduced for delivering niclosamide systemically.

### Niclosamide is a BCS Class II Drug, Thus Requiring Solubilization Technology

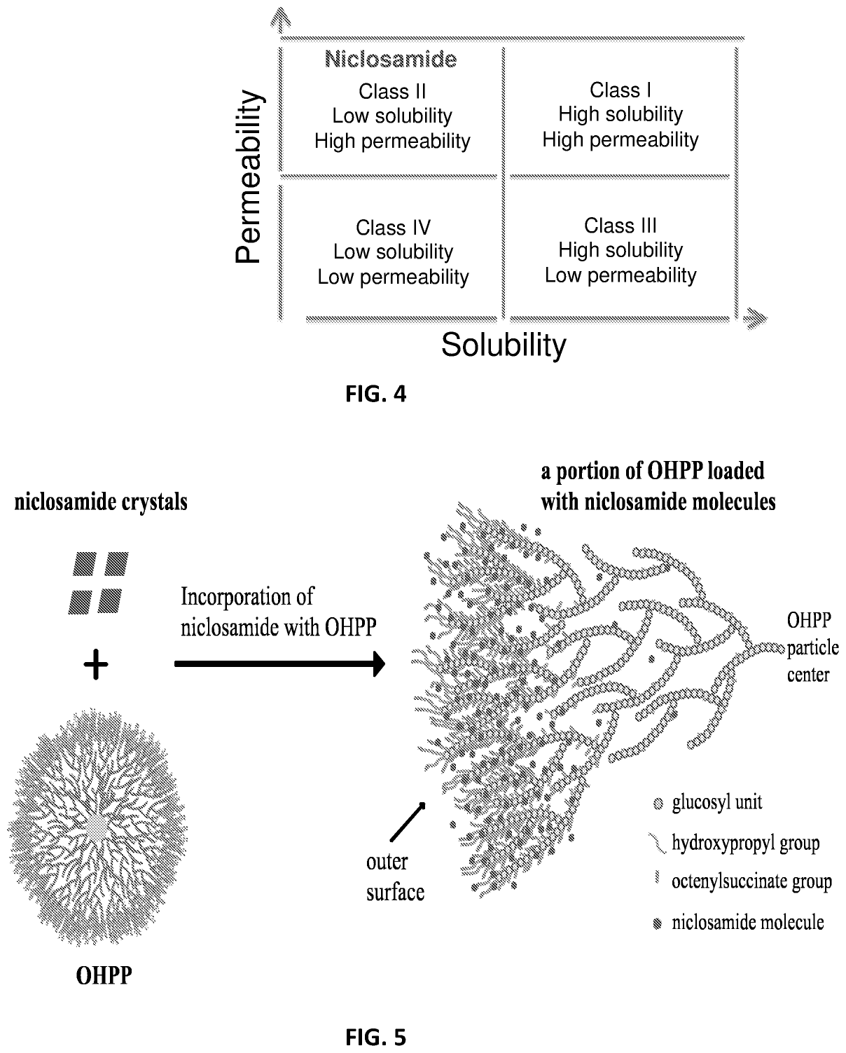

As shown in FIG. 4, the Biopharmaceutics Classification System (BCS) was established to predict the absorption of drugs. Based on the solubility and permeability, drugs can be classified in Class I (high solubility, high permeability), Class II (low solubility, high permeability), Class III (high solubility, low permeability), and Class IV (low solubility, low permeability).

As a BCS Class II drug, niclosamide has low solubility and high permeability (delMoral-Sanchez et al., 2019). Depending on the temperature of measurement, the solubility of niclosamide was about 0.3 to 1.14 μg/mL in phosphate buffer (Xie and Yao, 2018), and was defined as “practically insoluble” (delMoral-Sanchez et al., 2019). For Class II drugs, the most effective way to increase their absorption is to increase their solubility.

### Soluble Niclosamide Improves Bioavailability with Low Toxicity

Using single-capillary electrospray, Lin et al. (2016) prepared nanosuspension of niclosamide for increased solubility. To mice injected with tumor cells, niclosamide was orally administered at 100 mg/kg for 5 times a week and a total of 2 weeks. The results showed that the tumor weights were significantly reduced for treatment groups.

Meanwhile, they found that the niclosamide-treated group showed comparable body weight, nutrition levels (albumin), renal function, creatinine, and hepatic function as the control (non-treated) group. Also, the histopathological analysis of tissues from vital organs (brain, kidney, intestine, and liver) revealed no damage in either group. In addition, there were no significant differences in blood cells counts, hemoglobin, and platelet counts between the control and niclosamide-treated groups.

While showing improved bioavailability (25%) and low toxicity than regular niclosamide formulations, the disadvantages of electrosprayed niclosamide are its highly complicated preparation procedure and inability for production scale-up, as well as the use of toxic solvents (acetonitrile and DMSO) in preparation.

### Niclosamide to Treat COVID-19

COVID-19 shows lung thrombosis, frequent diarrhea, abnormal activation of the inflammatory response, and rapid deterioration of lung function consistent with alveolar oedema (Braga et al., 2021). The lungs of patients with COVID-19 contain infected pneumocytes with abnormal morphology and frequent multinucleation (Braga et al., 2021), and the generation of syncytia results from the activation of SARS-CoV-2 Spike protein at the cell plasma membrane level (Braga et al., 2021). With over 3000 approved drugs, Braga et al. (2021) performed screenings for inhibitors of Spike-driven syncytia while also focusing on drugs that also protected against virus replication and associated cytopathicity. They found that one of the most effective molecules was niclosamide, which markedly blunted calcium oscillations and membrane conductances in Spike-expressing cells by suppressing the activity of TMEM16F/Anoctamin6, a calcium-activated ion channel and scramblase responsible for phosphatidylserine exposure on the cell surface (Braga et al., 2021).

### DETAILED DESCRIPTION

### OHPP Improves the Solubility of Niclosamide

OHPP improves the solubility of poorly water-soluble drugs as shown with numerous model compounds such as celecoxib, paclitaxel, curcumin, and docetaxel. Niclosamide was used as a model due to its potential for repositioning as anti-cancer drug and its well-known difficulties to be solubilized. These results are reported in Xie and Yao (2018a, 2018b, 2019, 2020), the contents of which are incorporated herein by reference.

FIG. 5 shows the mechanism of OHPP to stabilize and solubilize niclosamide. Niclosamide alone exits in highly crystalized form with very low solubility (adapted from Xie and Yao et al. 2018b). OHPP is a nano-particulate with its core made of phytoglycogen, a dendrimer-like starch analogue isolated from a specialty corn. At the outer surface of phytoglycogen, there are two functional layers: (1) a layer of octenylsuccinate to provide hydrophobic interactions with drug molecules and to maintain highly solubility, and (2) a layer of hydroxypropyl layer to stabilize amorphous drug molecules through hydrogen bonds. Through the functional layers, OHPP maintains the stability of amorphous drug molecules not only in the solid state, but also when dispersed in water. In another word, OHPP-formulated niclosamide is stable in solid (e.g. in powder of tablet forms), and once it is dispersed in water, niclosamide are still stable (without rapid crystallization) to provide a sufficient time window (e.g. many hours) for absorption in the GI tract. Since niclosamide has high permeability, once it is dissolved, it is easily absorbed.

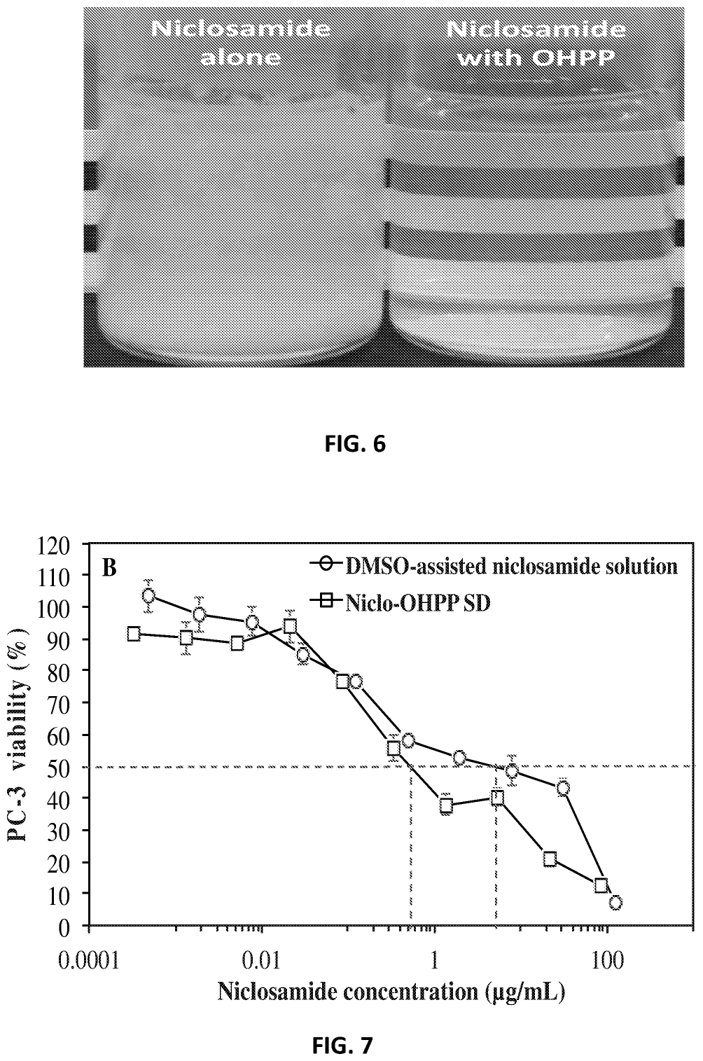

FIG. 6 compares the dispersion of niclosamide alone and OHPP-formulated niclosamide. For individual dispersions, the total amount of niclosamide was 2.5 mg/mL. Niclosamide alone did not dissolve and showed heavy clouds and precipitates. In contrast, the dispersion of OHPP-formulated niclosamide showed a high transparency, suggesting a high solubility. In fact, the solubility of OHPP-formulated niclosamide was over 5,000 times greater than that of niclosamide alone. In addition, the capability of OHPP to solubilize niclosamide was 50 times greater than that of HPMCAS (a leading solubilizer supplied by Ashland, Shin-Etsu Chemical, and Dow Chemical) and 300 times greater than that of Soluplus® (Xie and Yao, 2018b).

### OHPP-Formulated Niclosamide has High Bioaccessibility

The niclosamide solubilized by OHPP can be effectively released. Unlike DMSO and other solvents or technologies, the release of niclosamide from OHPP is gradual and therefore the recrystallization may be avoided during the absorption. FIG. 7 shows the inhibitory efficacy against a prostate cancer cell (PC-3) of niclosamide dissolved by DMSO or OHPP. For DMSO-dissolved niclosamide, the IC₅₀ was 5.93 μg/mL; for OHPP-dissolved niclosamide, the IC₅₀ was 0.68 μg/mL. OHPP thus performs better than DMSO in enabling niclosamide to provide biological effects (e.g. anti-cancer effect). DMSO-assisted solubilization is a conventional procedure in evaluating the in vitro efficacy of drug candidates, such as evaluating drugs for their anti-SARS-CoV-2 effects (Jeon et al., 2020).

### OHPP-Formulated Niclosamide has High Bioavailability

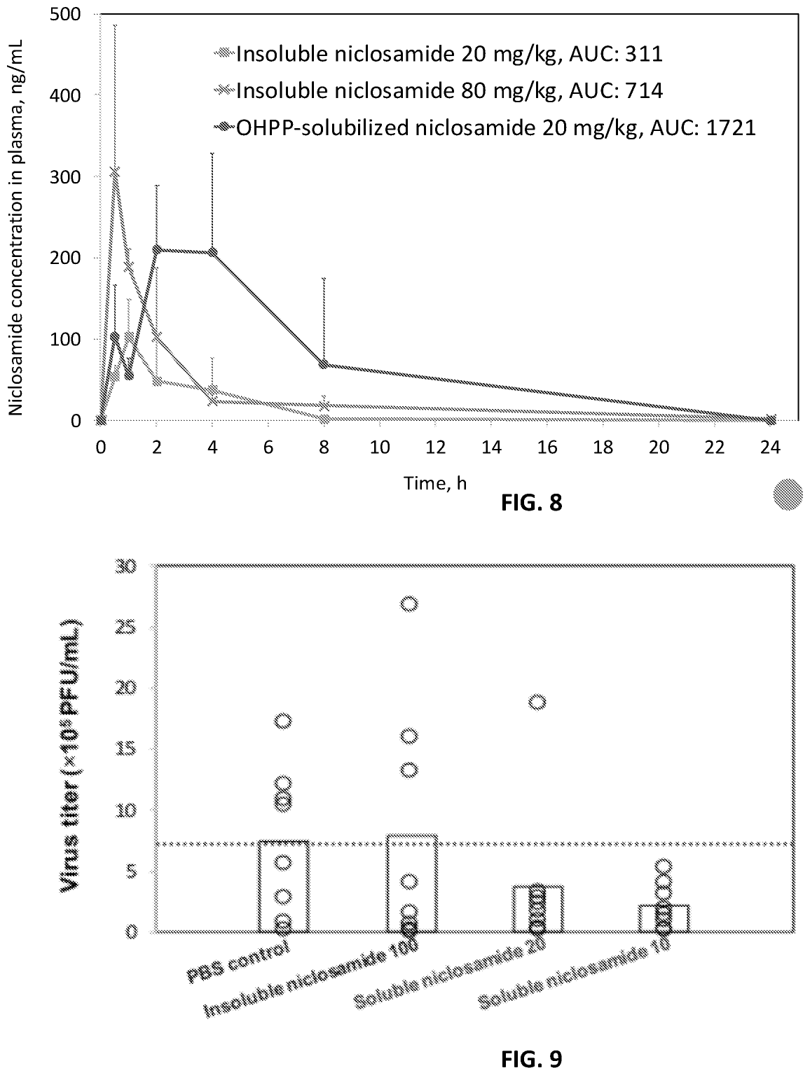

FIG. 8. Niclosamide concentration in plasma vs. time and “area under the curve (AUC)” for mice gavaged with insoluble niclosamide (20 or 80 mg/kg) or OHPP-formulated niclosamide (20 mg/kg).

The niclosamide solubilized by OHPP has much higher bioavailability than conventional niclosamide alone. FIG. 8 above shows niclosamide concentration in plasma vs. time and “area under the curve (AUC)” for mice gavaged with insoluble niclosamide (20 or 80 mg/kg) or OHPP-formulated niclosamide (20 mg/kg). Each data point is the mean of 3 replicate mice, with error bar as the standard deviation. The data indicated that niclosamide bioavailability increased by 5-10 times using OHPP formulation compared to insoluble niclosamide available in the market, much greater than other reported values to date. OHPP-solubilized niclosamide also resulted in prolonged retention in plasma of niclosamide level, which is highly beneficial to drug safety and efficacy.

### OHPP-Formulated Niclosamide Effective Against SARS-CoV-2

FIG. 9 shows lung virus titers of hACE2 transgenic mice at 5 days post infection. A study of SARS-CoV-2 challenged hACE2 transgenic mice was conducted in a BSL-3 lab. Four different treatment groups after infection were compared, including a control group treated with PBS, a group treated with insoluble niclosamide at 100 mg/kg, a group treated with OHPP-solubilized niclosamide at 20 mg/kg, and a group treated with OHPP-solubilized niclosamide at 10 mg/kg, twice a day for 5 days. The circles show the lung virus titer values of individual mouse, the bars are the means of individual treatment groups. The dash line at 7.5×10⁵ PFU/mL is used to indicate the level of lung virus titer over which a high lung viral load is considered to have occurred. The data shows that OHPP-solubilized niclosamide prevented or reduced the progression of initial infection to high viral load in lungs, while regular niclosamide at 5-10 times dose level did not show efficacy on viral load reduction.

### OHPP is a Modified Starch with Dendrimer-Like Nanostructure

OHPP was co-developed by Purdue and Phytoption using phytoglycogen as starting material. Phytoglycogen is a starch-type glucan isolated from a specialty corn grown in the US. As shown in FIG. 5, phytoglycogen and its derivatives show dendrimer-like nanostructure which provides unique functionalities. To generate OHPP, phytoglycogen is treated with methods broadly used in the food and pharmaceutical industries. Since phytoglycogen is a starch analogue, OHPP is a modified starch potentially with satisfactory safety profiles.

Niclosamide is a potential high-efficacy Covid-19 therapeutic, anticancer drug, and antiviral therapeutic for many other viral diseases. However, niclosamide is a BCS Class II drug with low solubility and high permeability. When used in large doses and for a long time, non-soluble niclosamide causes large variance in plasma concentration and side effects in the GI tract. In contrast, soluble niclosamide shows increased bioavailability and low toxicity. To effectively and practically solubilize the drugs that are difficult to be solubilized by current technologies, OHPP was developed using plant-based dendrimer-like starch material. In general, OHPP-formulated niclosamide increases solubility by over 5,000 times and realizes effective release of niclosamide molecules to provide biological efficacies. With the substantial increase of its solubility, the dose of niclosamide may be significantly reduced and the absorption may be greatly increased to achieve consistent antiviral efficacies at low toxicity. The strong in vitro bioaccessibility of OHPP-formulated niclosamide has been shown using cancer cell models.

The formulations of the disclosure may also be combined with other antiviral active ingredients such as other antiviral agents. In addition, the methods of the disclosure include treatment that also contains other active ingredients such as other antiviral, antibacterial, or anti-inflammation agents.

The following numbered embodiments are contemplated and are non-limiting:

Clause 1. A composition comprising highly branched glucan and niclosamide.

Clause 2 A composition comprising the highly branched glucan and niclosamide wherein the oral bioavailability of the niclosamide of said composition is at least 2 times higher than that of niclosamide alone.

Clause 3. The composition of clause 1-2 further comprising a pharmaceutically acceptable excipient.

Clause 4. The composition of clauses 1-3 in oral form.

Clause 5. The composition of clauses 1-4, wherein the composition is a solid.

Clause 6. The composition of clauses 1-4, wherein the composition is a pill, tablet, capsule, liquid, gel or powder.

Clause 7. The composition of clauses 1-3, wherein the composition is for inhalation, intranasal, intravenous, intramuscular, subcutaneous, or intradermal delivery.

Clause 8. The composition of clause 1 and 2 wherein the highly branched glucan is octenylsuccinate hydroxypropyl phytoglycogen (OHPP).

Clause 9. A method for treating virus infections comprising administering to a patient in need thereof an effective amount of a composition of clauses 1-7.

Clause 10. The method of clause 9 wherein the virus infection is caused by a virus selected from group consisting of SARS-CoV-2, SARS-CoV, MERS-CoV, Influenza A, Dengue virus, Zika virus, Ebola virus, Hepatitis C virus, Japanese encephalitis virus, Human rhinoviruses, Chikungunya virus, Human adenovirus, Epstein-Barr virus, other Influenza, HIV, Polio virus, other viruses, and their combinations.

Clause 11. A method for treating cancer comprising administering to a patient in need thereof an effective amount of a composition of clauses 1-7.

Clause 12. The method of clause 11, wherein the cancer is prostate cancer, Adrenocortical carcinoma, Breast cancer, Colon cancer, Glioma, Head and neck cancer, Leukemia, Lung cancer, Osteosarcoma, Ovarian cancer, Prostate cancer, Renal cell carcinoma, or ovarian cancer.

Clause 13. The method of clauses 9-12, wherein the patient is an animal.

Clause 14. The method of clause 9-12, wherein the patient is a human.

Clause 15. The method of clauses 9, 13, and 14 wherein the virus is SARS-CoV-2.

Clause 16. A method of preventing viral infection in patient comprising the step of administering to patient an effective amount of a composition of clauses 1-7.

Clause 17. The method of clause 16, wherein the patient is human.

Clause 18. The method of clauses 16-17 wherein the viral infection is caused by a virus selected from group consisting of SARS-CoV-2, SARS-CoV, MERS-CoV, Influenza A, Dengue virus, Zika virus, Ebola virus, Hepatitis C virus, Japanese encephalitis virus, Human rhinoviruses, Chikungunya virus, Human adenovirus, Epstein-Barr virus, other Influenza, HIV, Polio virus, other viruses, and their combinations.

Clause 19. A composition comprising the composition of clauses 1-7 and one or more additional active agents.

Clause 20. The composition of clause 19, wherein the one or more additional active agents are antiviral agent, antibacterial agent, antifungal agent, and/or anti-inflammation agent.

Clause 21. A method for treating virus infection comprising administering to a patient in need thereof an effective amount of a composition of clauses 1-7 and further administering an effective amount of one or more additional active agents.

Clause 22. The method of clause 21, wherein the one or more additional active agents are antiviral agent, antibacterial agent, antifungal agent, and/or anti-inflammation agent.

Clause 23. The method for treating cancer comprising administering to a patient in need thereof an effective amount of composition of clauses 1-7 and further administering an effective amount of one or more additional active agents.

Clause 24. The method of clause 23, wherein the one or more additional active agents are anticancer agents.

Clause 25. A method for treating a disease or disorder comprising administering to a patient in need thereof an effective amount of a composition of clauses 1-7, wherein the disease or disorder is a bacterial infection caused by tuberculosis, anthrax, pseudomonas aeruginosa, or staphylococcus aureus, a type 2 diabetes mellitus, nonalcoholic fatty liver disease, artery constriction, endometriosis, neuropathic pain, rheumatoid arthritis, sclerodermatous graft-versus-host disease, or a systemic sclerosis.

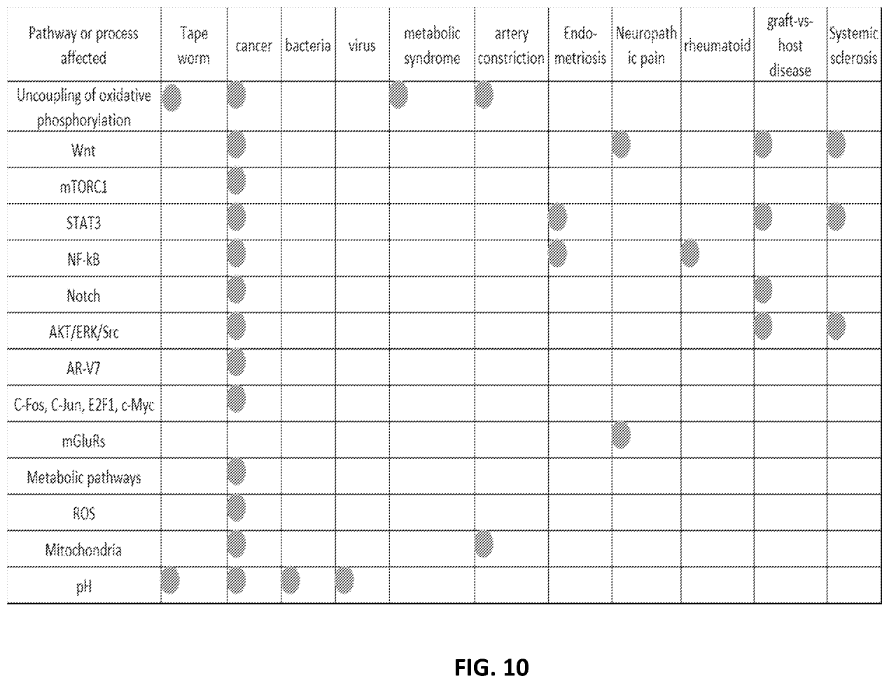

Clause 26. A method for targeting one of more signaling pathways and biological processes indicated in the table below comprising administering to a patient in need thereof an effective amount of composition of clauses 1-7 (See FIG. 10 for more details).

Clause 27. The composition of clause 1, where the highly branched glucan and niclosamide are incorporated using a method selected from the group consisting of spray drying, spray chilling, extrusion, hot-melt extrusion, homogenization, milling such as jet milling, cryo-milling, ball milling, and colloid milling, vacuum drying, drum drying, film-forming, surface coating, printing, 3D printing, multi-layer forming, high-intensity blending, co-crystallization, personalized manufacturing of dosage forms, and their combinations.

### EXAMPLE: PREPARATION OF OHPP-FORMULATED ICLOSAMIDE

OHPP-formulated niclosamide can be prepared through combining OHPP and niclosamide in a physical approach. For example, niclosamide and OHPP were dissolved in ethanol and spray-dried to collect the solid. Three grams of niclosamide and 9.0 g OHPP were both dissolved in 600 mL ethanol, and the solution was spray-dried using a Büchi mini-spray dryer B-290 (BUCHI, Switzerland) equipped with nitrogen purge for use with organic solvents. The inlet and outlet temperatures were 90 ° C. and 57-60 ° C., respectively. The feed rate was 6 mL/min with nitrogen gas flow rate of 350 L/h. The solvent in the nitrogen gas from the outlet was condensed and collected using Büchi Inert Loop B-295.

Niclosamide and OHPP can be incorporated through other physical procedures, either with or without spray-drying. These procedures include but are not limited: (1) extrusion such as hot-melt extrusion, (2) homogenization, (3) jet milling, (4) cryo-milling, (5) ball milling, (6) vacuum drying or drum-drying, (7) film-forming or surface coating, (8) printing, (9) 3D printing, (10) multi-layer forming, (11) spray-chilling, (12) personalized manufacturing of dosage forms, (13) high-intensity blending, (14) co-crystallization, and (15) the combination of different procedures.

### EXAMPLE: IN VIVO PHARMACOKINETIC STUDY

The in-vivo studies were performed at the Purdue Translational Pharmacology Facility. Male B6.Cg-Tg(K18-ACE)2Prlmn/J NCAR Non-carrier mice (8-10 weeks) were obtained from Jackson Laboratories, (Bar Harbor ME) for the study. Animals were group housed and handled according to institutional guidelines as per protocol 1405001069 Purdue University. Animals were kept in a temperature controlled environment with a 12 h light/dark cycle and received standard diet and water ad libitum.

Animals were dosed per oral with different dose of niclosamide: 1) A group of animals were dosed with 80 mg/kg of insoluble niclosamide suspension, 2) a group of animals were dosed with 20 mg/kg of insoluble niclosamide suspension, and 3) a group of animals were dosed with 20 mg/kg of OHPP-formulated niclosamide solution, and at time of blood collection were sedated with isoflurane and exsanguinated via cardiac stick. Blood was placed into EDTA tubes (Mini-Collect, Greiner Bio-One) and centrifuged for 10 min at 4500 rpm. Upon processing plasma was immediately frozen in dry ice and stored at −80 ° C. until time of analysis.

FIG. 8 shows niclosamide concentration in plasma vs. time and “area under the curve (AUC)” for mice gavaged with insoluble niclosamide (20 or 80 mg/kg) or OHPP-formulated niclosamide (20 mg/kg). Each data point is the mean of 3 replicate mice, with error bar as the standard deviation.

### EXAMPLE: IN VIVO SARS-COV-2 CHALLENGE STUDY

Animal experimental protocols were reviewed, approved, and supervised by the Institutional Animal Care and Use Committee at the University of Chicago. All infections were carried out in animal biological safety level-3 containment laboratories at the Howard Taylor Ricketts Laboratory. Mice were housed in cages with HEPA filters. 6-8 weeks old female and male B6.Cg-Tg(K18-ACE2)2Prlmn/J (K18-hACE2) mice were obtained from Jackson Laboratory. Animals were challenged with 2×10⁴ PFU of USA-WA1/2020 SARS-CoV-2 (2019-nCoV) in 20 μ1 by intranasal injection. Following challenge, animals were treated every 12 hours with either PBS or niclosamide drugs. At day five post-challenge, all animals were killed and subjected to necropsy to remove the lungs. Lungs were homogenized in 2% DMEM to measure viral titers.

Viral samples were serially diluted 10-fold and used to infect 4-wells of VeroE6 cells for 1 hour, inoculum was removed and warm carboxymethylcellulose/media was added and incubated for 3 days. Overlay media was removed and plates were fixed in 10% formalin for 30 min and stained with crystal violet for 1 hour and subsequently plaques were counted. Following the count, plaque forming units per milliliter were calculated for the original sample.

FIG. 9 shows lung virus titers of hACE2 transgenic mice at 5 days post infection. Four different treatment groups after infection are compared, including a control group treated with PBS, a group treated with insoluble niclosamide at 100 mg/kg, a group treated with OHPP-solubilized niclosamide at 20 mg/kg, and a group treated with OHPP-solubilized niclosamide at 10 mg/kg, twice a day for 5 days. The circles show the titer values of individual mice, the bars are the means of individual treatment groups. The dash line at 7.5×10⁵ PFU/mL is used to indicate the level of lung virus titer over which a high lung viral load is considered to have occurred.

### PROPHETIC EXAMPLES

As a general approach, we will conduct pharmacokinetic and toxicological studies of niclosamide formulations for oral uses. The formulations contain OHPP, niclosamide, with or without other pharmaceutically acceptable excipients.

In addition, we will conduct infected animal efficacy studies and/or human trials. The overall consideration is that OHPP-formulated niclosamide, through enhanced niclosamide solubility, can be used to achieve desirable and consistent plasma concentration and therapeutic efficacy with an acceptable safety profile.

### Prophetic Example 1

Virus and cells: A SARS-CoV-2 strain, NMC-nCoV02, will be propagated in Vero cells in Dulbecco's modified Eagle medium (DMEM; Gibco, Grand Island, NY) supplemented with 1% penicillin-streptomycin (Gibco) and TPCK (tosylsulfonyl phenylalanyl chloromethyl ketone)-treated trypsin (0.5 g/ml; Worthington Biochemical, Lakewood, NJ) in a 37° C. incubator supplemented with 5% CO₂ for 72 h. Propagated virus is stored at −80° C. as the working virus stock for animal studies. The 50% tissue culture infective dose (TCID50) is determined through fixation and crystal violet staining.

Control: A group with no infection is used as control (control group).

Infection: 3 groups of male ferrets (8/group), seronegative for SARS-CoV-2 are intranasally inoculated with 10S8 TCID5o/m1 of NMC-nCoV02 under anesthesia.

Niclosamide treatments: For treatment, group-1 (single daily dose) of animals (8/group) is administered with OHPP-formulated niclosamide at 5 mg/kg once daily, and group-2 (double daily dose) (8/group) with the same formulation at 5 mg/kg twice daily, all by oral gavage, starting at 1 day post-infection (dpi) and continuing through 10 dpi. For control group, PBS is administered to 8 animals (PBS group).

Pathological monitoring & test: Nasal washes and stool specimens are collected every day. Virus titers in nasal washes and tissues are determined using 50% TCID₅₀ assessment in Vero cells; virus titers in stool specimens are measured using quantitative real-time PCR (qRT-PCR). Briefly, total RNA is extracted using TRIzol reagent (Thermo Fisher Scientific) or an RNeasy kit (Qiagen), and cDNAs are generated with a SARS-CoV-2-specific primer by reverse transcription using QuantiTect reverse transcription (Qiagen). qRT-PCRs are performed using a SYBR green supermix (Bio-Rad) and a CFX96 Touch real-time PCR detection system (Bio-Rad) with a spike gene-based, SARS-CoV-2-specific primer set, and virus RNA copy numbers are calculated as a ratio with respect to the PBS group (infected but not treated by niclosamide). Four animals per group are euthanized at 5 dpi and the other four euthanized at 12 dpi, and nasal turbinate and lungs were collected to measure tissue virus titers and examine lung histopathology.

Statistical analysis: Statistical analyses will be conducted to assess significant differences in values for weight loss, temperature, and viral titers.

### EXAMPLE 2

Animals:
Species: Hamster (Syrian golden)
Groups: 6×2 for PK study, 4+8×3 for efficacy study
Sex: all Male

Pharmacokinetic (PK) study:
Two doses, 6 for each dose
For each animal, 4 blood samples are collected
Two sub-groups for each dose, each sub-group (3) contribute to 4 time points. 8 time points in total for each dose
Plasma samples collected, cold-stored, and analyzed for niclosamide quantification
PK data used to define doses for efficacy study

Efficacy study:
Four groups: normal healthy group (4); infected/non-treated group (8); and two infected/niclosamide-treated groups (8×2)
Health group: all (4) sacrificed at mid-point for lung analysis
Infected/non-treated group: half (4) sacrificed at mid-point, half (4) sacrificed at end point

Two infected/treated groups:
Group-1: double dose daily, gavage, low dose
Group-2: double dose daily, gavage, high dose
Half (4) sacrificed at mid-point, half (4) sacrificed at end point

Mid-point: to allow for maximal lung symptoms (day 5)

End point: to allow for weight and temperature to recover (day 12)

Data collected:
Each day: weight, temperature, nasal wash & feces—for viral load analysis
Mid-point: 4 from each of four groups—sacrificed for lung viral load & histopathological analysis
End point: 4 for each of three infected groups—sacrificed for lung viral load & histopathological analysis

The PK study provides key information of: (1) the doses needed to reach a desirable plasma concentration, (2) bioavailability of niclosamide, and (3) kinetic parameters including the time to reach the maximal plasma concentration for infected animal efficacy studies.

### EXAMPLE 3

A preliminary toxicity and toxicokinetic (TK) study are conducted with elevated doses. Below is a study design of repeat dose study of OHPP formulated niclosamide in Sprague Dawley Rats

TABLE 1
7-Day Dose-Range Finding Study of OHPP formulated Niclosamide in Rats.
DRF (dose range finding) Study in Rats

| | |
|---|---|
| GLP Status | Non-GLP |
| Acclimation Period | 1 Week |
| In-Life Period | 1 week, not including acclimation period |
| Number of Animals/Sex/Group (Main) | 5/sex/group |
| Number of Animals/Sex/Group (TK) | 6/sex/group in treated groups |
| Number of Groups (including Control) | 5 dose groups (including control, and four |
| | dose levels) |

Formulations

| | |
|---|---|
| Formulation preparation | Once daily or if stable as needed basis |
| Dose Frequency/Route of administration | Daily for 7 days/Oral |

Observations

| | |
|---|---|
| Cage Side Observations | At least once/day |
| Clinical Observations | Daily |
| Body Weights | pre dose, Days 1, 3 and 7 |
| Food Consumption | quantitative Days 1 to 3 and 3 to 7 |
| Ophthalmology | N/A |

Clinical Pathology and TK

| | |
|---|---|
| Clinical Chemistry, Hematology, Coagulation | Prior to termination |
| Urinalysis | Prior to termination |
| TK Collection | 3/sex in the control group to be bled at one |
| | time point after dosing on SD 1 and 7. |
| | Cohorts of 3/sex/treated group to be bled at 8 |
| | time points after the first and last dose. |
| TK Samples | Samples to be sent for analysis |
| TK Data Evaluation | TK report prepared by CRO |

Post in live phase

| | |
|---|---|
| Necropsy/Tissue Preservation | 24 hrs after last dose - Full necropsy, full |
| | tissue collection and standard organ weights |
| | on all animals in all groups. |
| Bone Marrow | Collected. Evaluated upon request |
| Histopathology | Selected organs (Stomach, duodenum, |
| | kidneys, liver, lung and heart and any organ |
| | with gross lesions) |

DRF Study - animals necropsied 24 hrs following last dose

| | | | | | Dose |
|---|---|---|---|---|---|
| | | Number of Animals | | Dose Level | (mg/mL) |
| Group | Subgroups | Male | Female | (mg/kg/day) | concentrations |
| 1 (Saline | 1 (Toxicity) | 5 | 5 | 0 | |
| Control) | | | | | |
| 2 Low | 1 (Toxicity) | 5 | 5 | 50 | TBD |
| | 2 (Toxicokinetics) | 6 | 6 | | |
| 3 Mid | 1 (Toxicity) | 5 | 5 | 150 | TBD |
| | 2 (Toxicokinetics) | 6 | 6 | | |
| 4 High | 1 (Toxicity) | 5 | 5 | 500 | TBD |
| | 2 (Toxicokinetics) | 6 | 6 | | |
| 5 OHHP | 1 (Toxicity) | 5 | 5 | 1500 | TBD |

### EXAMPLE 4

One month repeat dose toxicity and toxicokinetic study with 2 weeks reversibility in rats (GLP)
In vitro Bacterial Mutation (Ames) assay
In vitro Human Lymphocyte Assay

As highlighted above, viral infections such as the current COVID-19 pandemic present an eminent health threat. While most of the efforts to improve the solubility of niclosamide have focused on oncology targets, there is evidence suggesting that niclosamide may target the virus directly (Chen et al, 2018, Lou et al, 2011). In the search for potential agents for host defense in viral infections, niclosamide has repeatedly stood out when screening chemical libraries of marketed drugs, e.g., Severe Acute Respiratory Syndrome coronavirus (SARS-CoV) (Wu et al, 2004); Chikungunya virus (Wang et al, 2016); Zika virus (Xu et al, 2016). See Chen et al (2018) for more details. The mechanism of action may be related to niclosamide's protonophore activity and its ability to act as a proton carrier (Staples, et al, 2009). Thus, whether through in vitro screening of known viruses or using virus models, niclosamide appears to provide antiviral properties that may prove beneficial in the treatment of patients with COVID-19, with an appropriate systemic dose.

### EXAMPLE 5; CLINICAL DEVELOPMENT PLAN

Phytoption plans to initiate a Phase 1 clinical trial to evaluate the safety and pharmacokinetics (PK) of NIC-OHPP-101 (an OHPP-formulated niclosamide) in patients with mild to moderate COVID-19. The trial will enroll 6 patients per cohort in a dose escalation manner for 5 cohorts in total. The starting dose in the Phase 1a clinical trial will be determined based on the nonclinical toxicology and toxicokinetic data. A Phase lb study in the same patient population (mild-to-moderate COVID-19) will assess one or more of the Phase 1a doses in more patients.

Study Population: The proposed study population are patients with mild to moderate COVID-19. The key criteria defining these populations are as follows: Patients that present with the following symptoms will be defined with “Mild illness”:
Increased temperature
a dry cough
tiredness
feeling slightly breathless
muscle pain
headache
sore throat
diarrhea

Patients with “Moderate illness” are more breathless and tend to have an increased heart rate, particularly if they're moving around. In addition to the symptoms defined for mild illness above, moderate illness includes:
a temperature higher than 37.8° C. (100.04° F.)
breathless during moderate exercise (e.g., walking upstairs)
soreness from coughing, but no pain
more persistent cough, several times an hour
headache
tiredness and marked lethargy
dry mouth

Objectives & Endpoints: The objectives of this study are to determine if oral formulations of niclosamide-OHPP are safe for patients with mild to moderate COVID-19 and evaluate the systemic pharmacokinetics of various doses of NIC-OHPP-101. While the primary endpoints will assess the safety and PK of NIC-OHPP-101, key secondary endpoints will measure the treatment effect on the underlying COVID-19 infection at Days 7, 14 and 28.

The primary objective is to evaluate the safety of oral NIC-OHPP-101 and determine if treatment with NIC-OHPP-101 decreases the SARS-CoV-2 viral load in patients with COVID-19 as determined by change in respiratory viral clearance using oropharyngeal swab or sputum samples on Day 7 and Day 14. Key secondary objectives in this study are: Cumulative incidence of hospitalizations (at Day 14); Cumulative incidence of the use of oxygen therapy, non-invasive ventilation or invasive ventilation (at Day 14)

Other objectives in this study are Mortality at Day 14 and Day 28

Safety objectives: The safety of NIC-OHPP-101 will be assessed via the reporting of treatment emergence adverse events (TEAEs), changes in clinical laboratory tests, vital signs and physical examinations.

Dose Justification: The current dose of niclosamide is up to 2 grams per day as an antihelminthic treatment. This high dose still results in minimal absorption in systemic circulation. It is anticipated that increased absorption with the formulations of disclosure allow for a controlled level of systemic exposure and concomitant treatment of Covid-19. Planned nonclinical studies will provide guidance in selecting a first-in-human dose based on an appropriate safety margin. The goal of Phase 1a dose escalation is to establish no more than 2 doses for evaluation in Phase 1b to determine a minimally effective dose.

Dose Selection: The starting dose and dose escalation doses will be determined based on the GLP nonclinical toxicology studies.

Duration of treatment: It is anticipated that the duration of daily treatment will be 7 days, with continued monitoring for a total of 28 days.

### EXAMPLE 6 STUDY SYNOPSIS

### Protocol Concepts

Below is a phase I synopsis for one of the drugs, it would apply to other OHPP-formulated niclosamide drugs as well.

NIC-OHPP-101 Phase 1 Synopsis

Name of Sponsor/Company: Phytoption LLC Name of Investigational Product:

NIC-OHPP-101 Name of Active Ingredient: Niclosamide

Study center(s): One investigational center will participate

Title of Study: A Phase 1 Randomized, Double-Blind, Controlled Study to Evaluate the Safety and Efficacy of NIC-OHPP-101 in Patients with Mild to Moderate COVID-19.

Number of patients: Approximately 60-90 patients will be enrolled. Phase 1a: 30 patients equally evaluated in 5 ascending doses, Phase 1b: 30 patient expansion in up to 2 dose levels

Phase of development: 1

Objectives:

The primary objective is: To evaluate the safety of oral NIC-OHPP-101 and determine if treatment with NIC-OHPP-101 decreases the SARS-CoV-2 viral load in patients with COVID-19 as determined by change in respiratory viral clearance using oropharyngeal swab samples on Day 7 and Day 14.

Key secondary objectives in this study are: Cumulative incidence of hospitalizations (at Day 14); Cumulative incidence of the use of oxygen therapy, non-invasive ventilation or invasive ventilation (at Day 14)

Other objectives in this study are: Mortality at Day 14 and Day 28

Safety objectives: The safety of NIC-OHPP-101 will be assessed via the reporting of treatment emergence adverse events (TEAEs), changes in clinical laboratory tests, vital signs and physical examinations.

Study Design and Duration

Potential patients with COVID-19 will be screened with a quantitative real-time polymerase chain reaction (q-rtPCR)-based assay.

Phase 1a: Patients that demonstrate mild or moderate signs of infection will be enrolled into the Phase 1a study. Six patients will be enrolled into five ascending dose cohorts to slowly evaluate the safety of NIC-OHPP-101 and determine two potential doses that provide appropriate safety and efficacy on viral shedding. If the first six patients do not demonstrate any clinically significant adverse effects by Day 7 the second cohort will be enrolled. Additional cohorts will be subsequently enrolled following the safety assessment of the preceding dose cohort on Day 7 of treatment. Patients in all cohorts will receive 7 days of dosing and final monitoring of patients will occur on Day 28.

Phase 1b: The Phase 1a part of this clinical trial will determine up to 2 dose levels to use in the cohort-expansion patients with COVID-19 to further evaluate safety and efficacy of NIC-OHPP-101. Thirty (30) additional patients per cohort with asymptomatic, mild or moderate COVID-19 infections will be enrolled in this 28-day phase. The primary endpoint assessment, in addition to safety, is the reduction in respiratory oropharyngeal viral load by Day 7 and Day 14. All patients in this Phase 1b clinical trial will be evaluated over the course of 28 days.

Investigational product, dosage and mode of administration:

NIC-OHPP-101 contains niclosamide and octenylsuccinate hydroxypropyl phytoglycogen in a capsule form for oral delivery. The ratio of niclosamide to OHPP is 25:75 and is formulated for once-daily dosing.

Study Population:

Key Inclusion Criteria: Age 18 years and older; Positive SARS-Cov-2 (q-rtPCR) test on oropharyngeal swab; No requirement for hospitalization or oxygen therapy at time of enrollment; Women of child-bearing potential and men must agree to use adequate contraception (hormonal or barrier method of birth control; abstinence) prior to study entry, for the duration of study participation, and for 7 days following completion of therapy. NOTE: Should a woman become pregnant or suspect she is pregnant while participating in this study, she should inform her treating physician immediately. Women of child-bearing potential should use highly effective methods of birth control. These are those methods of contraception, alone or in combination, that result in a low failure rate (i.e, less than 1% per year) when used consistently and correctly; Ability to sign the informed consent document

Key Exclusion Criteria:

Evidence of respiratory failure, sepsis, organ dysfunction/failure; Moderate disease with risk factor(s): peripheral capillary oxygen saturation (SpO2) >92% on room air with one of the following risk factors for development of severe disease: a. Age>60 years, b. Onmedicationforhypertension, c. Diagnosed diabetes mellitus, d. Knowncardiacdisease, e. Obesity(BMI>35 kg/m2), f. Active malignancy, g. Immunosuppression(usingbiologicsorglucocorticoids>20mg/dprednisine equivalent for >2 weeks); Pregnancy or breast feeding; Hypersensitivity to any of the components of NIC-OHPP-101

Statistical methods: Descriptive statistics will be used to evaluate the safety aspects of NIC-OHPP-101.

Determining a statistically significant reduction in viral load in the Phase 1b cohorts will employ the appropriate statistical tests.

We expect that OHPP-formulated niclosamide can be used to treat or prevent SARS-CoV, SARS-CoV-2, Zika, MERS, Dengue, Influenza, Chikungunya virus, HIV, Ebola, Polio, or other viruses; and also treat cancers including prostate cancer, adrenocortical carcinoma, breast cancer, colon cancer, glioma, head and neck cancer, leukemia, lung cancer, osteosarcoma, ovarian cancer, prostate cancer, renal cell carcinoma, or ovarian cancer; and also bacterial infection caused by tuberculosis, anthrax, pseudomonas aeruginosa, or staphylococcus aureus, or diseases or illness such as type 2 diabetes mellitus, nonalcoholic fatty liver disease, artery constriction, Endometriosis, neuropathic pain, rheumatoid arthritis, sclerodermatous graft-versus-host disease, or a systemic sclerosis. Similar animal efficacy studies and/or clinical trials will also be carried out with animals or patients for those diseases.

Since OHPP-formulated niclosamide can also be made into other drug forms, such as inhalation, nasal spray cream, lotion, injection, etc. we will conduct pharmacokinetic, efficacy and toxicological studies of those OHPP-formulated niclosamide dosage forms as therapeutic treatments, and also for prophylactic uses against viral infections. The administration routs include but not limited to oral route, such as pill, tablet, capsule, liquid, powder, film, gel, etc, powders or liquid for inhalation or intranasal delivery, injections including but not limited to intravenous, intramuscular, subcutaneous, or intradermal delivery.

In addition to being a human drug, niclosamide has long been used in animals to treat worms. Many animals including food animals and companion animals can be infected by viruses, such as swine flu, avian flu, SARS-CoV-2, Peste des petit ruminants, Bluetongue, AHS (African horse sickness), AHSV (African horse sickness virus), AIDS (acquired immunodeficiency syndrome), ASF (African swine fever), BSE (Bovine spongiform encephalopathy), BT (bluetongue virus), CMLV (camelpox virus), CSF (Classical swine fever), CSFV (classical swine fever virus), enJSRV (endogenous JSRV-related retroviruses), FMD (foot-and-mouth disease), FMDV (foot-and-mouth disease virus), GTPV (goatpox virus), HA (hemagglutinin), HIV (human immunodeficiency virus), HTLV (human T cell leukemia virus), JSRV (jaagsiekte sheep retrovirus), MVV (Maedi-visna virus), NDV (Newcastle disease viruses), OPA (ovine pulmonary adenocarcinoma), RSV (Rous sarcoma virus), SARS (severe acute respiratory syndrome), SARS-CoV (SARS-Coronavirus), SPV (sheeppox virus), TB (tuberculosis), VARV (variola virus), etc. OHPP-formulated niclosamide can also be animal drugs to treat above diseases.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCE CITED

1. Beigel J H, Tomashek K M, Dodd L E, et al. (2020). Remdesivir for the treatment of Covid-19—preliminary report. The New England Journal of Medicine. https://www.nejm.org/doi/full/10.1056/NEJMoa2007764
2. Braga L, Ali H, Secco I, Chiavacci E, Neves G, et al. 2021. Drugs that inhibit TMEM16 proteins block SARS-CoV-2 Spike-induced syncytia. Nature, https://doi.org/10.1038/s41586-021-03491-6.
3. Chang Y W, Yeh T K, Lin K T et al. (2006). Pharmacokinetics of anti-SARS-CoV agent niclosamide and its analogs in rats. Journal of Food and Drug Analysis, 14: 329-333. https://www.researchgate.net/profile/Chiung-Tong_Chen/publication/228738372_Pharmacokinetics_of_Anti-SARS-CoV_Agent_Niclosamide_and_Its_Analogs_in_Rats/links/00b7d51428aa7cd584000000.
4. Del Moral-Sanchez J M, Gonzalez-Alvarez I, Gonzalez-Alvarez M, Navarro A, Bermejo M (2019). Classification of WHO essential oral medicines for children applying a provisional pediatric biopharmaceutics classification system. Pharmaceutics, 11: 567. https://www.mdpi.com/1999-4923/11/11/567
5. Gassen N C, Papies J, Bajaj T, et al. (2020). Analysis of SARS-CoV-2-controlled autophagy reveals spermidine, MK-2206, and niclosamide as putative antiviral therapeutics. bioRxiv preprint, https://www.biorxiv.org/content/10.1101/2020.04.15.997254v1
6. Jeon S, Ko M, Lee J, et al. (2020). Identification of antiviral drug candidates against SARS-CoV-2 from FDA-approved drugs. Antimicrobial Agents and Chemotherapy, https://aac.asm.org/content/early/2020/04/28/AAC.00819-20
7. Li Y, Li P K, Roberts M J, et al. (2014). Multi-targeted therapy of cancer by niclosamide: A new application for an old drug. Cancer Letters, 349: 8-14. https://www.sciencedirect.com/science/article/abs/pii/S0304383514002092?via%3Dihub
8. Lin C K, Bai M Y, Hu T M, et al. (2016). Preclinical evaluation of a nanoformulated antihelminthic, niclosamide, in ovarian cancer. Oncotarget, 7: No. 8. https://www.oncotarget.com/article/7113/text/
9. Schweizer M T, Haugk K, McKiernan J S, et al. (2018). A phase I study of niclosamide in combination with enzalutamide in men with castration-resistant prostate cancer. PLOS ONE, https://journals.plos.org/plosone/article?id=10.1371/journal.pone.0198389
10. Watson J et al. (2020). Open letter to M R Mehra, S S Desai, F Ruschitzka, and A N Patel, authors of “Hydroxychloroquine or chloroquine with or without a macrolide for treatment of COVID-19: a multinational registry analysis”. Lancet. 2020 May 22:SO140-6736(20)31180-6. doi: 10.1016/S0140-6736(20)31180-6. PMID: 32450107 and to Richard Horton (editor of The Lancet). Concerns regarding the statistical analysis and data integrity. https://zenodo.org/record/3864691#.XtV69i2ZMW8
11. World Health Organization (WHO). (2019). World Health Organization Model List of Essential Medicines, 21st List, 2019. https://apps.who.int/iris/bitstream/handle/10665/325771/WHO-MVP-EMP-IAU-2019.06-eng.pdf?ua=1
12. Xie Y, Yao Y. (2018a). Octenylsuccinate hydroxypropyl phytoglycogen, a dendrimer-like biopolymer, solubilizes poorly water-soluble active pharmaceutical ingredients, Carbohydrate Polymers, 180: 29-37
13. Xie Y, Yao Y. (2018b). Octenylsuccinate hydroxypropyl phytoglycogen enhances the solubility and in-vitro antitumor efficacy of niclosamide. International Journal of Pharmaceutics, 535: 157-163
14. Xie Y, Yao Y. (2019). Incorporation with dendrimer-like biopolymer leads to improved soluble amount and in vitro anticancer efficacy of paclitaxel. Journal of Pharmaceutical Sciences, 108: 1984-1990
15. Ying Xie, Yuan Yao. (2020). Preparation and characterization of a solid dispersion containing curcumin and octenylsuccinate hydroxypropyl phytoglycogen for improved curcumin solubility. European Journal of Pharmaceutical Sciences
16. Xu J, Shi PY, Li H, Zhou J. (2020). Broad spectrum antiviral agent niclosamide and its therapeutic potential. ACS Infectious Diseases, 6: 909-915. https://pubs.acs.org/doi/10.1021/acsinfecdis.0c00052.

1. A composition comprising a highly branched glucan and niclosamide, or a pharmaceutically acceptable salt, hydrate, or solvate thereof.

2. The composition according to claim 1, wherein the oral bioavailability of niclosamide of said composition is at least 2 times higher than that of niclosamide alone as a pharmaceutical composition.

3. The composition of claim 1, further comprising one or more pharmaceutically acceptable excipients or diluents.

4. The composition of claim 1, wherein said composition is formulated for oral delivery.

5. The composition of claim 2, further comprising one or more absorption enhancers for further improved oral bioavailability of niclosamide.

6. The composition of claim 1, wherein the composition is a pill, tablet, capsule, liquid, gel or powder.

7. The composition of claim 1, wherein the composition is for inhalation, intranasal, intravenous, intramuscular, subcutaneous, or intradermal delivery.

8. The composition of claim 1, wherein the highly branched glucan is octenylsuccinate hydroxypropyl phytoglycogen (OHPP).

9. A method for treating or preventing a virus infection comprising administering to a patient in need thereof an effective amount of a composition of claim 1.

10. The method of claim 9, wherein the virus infection is caused by a virus selected from the group consisting of SARS-CoV-2, SARS-CoV, MERS-CoV, Influenza A, Dengue virus, Zika virus, Ebola virus, Hepatitis C virus, Japanese encephalitis virus, Human rhinoviruses, Chikungunya virus, Human adenovirus, Epstein-Barr virus, other Influenza, HIV, and Polio virus.

11. A method for treating cancer comprising administering to a patient in need thereof an effective amount of a composition of claim 1.

12. The method of claim 11, wherein the cancer is prostate cancer, adrenocortical carcinoma, breast cancer, colon cancer, glioma, head and neck cancer, leukemia, lung cancer, osteosarcoma, ovarian cancer, prostate cancer, renal cell carcinoma, or ovarian cancer.

13. The method of claim 9, wherein the patient is an animal.

14. The method of claim 9, wherein the patient is a human.

15. The method of claim 9, wherein the virus is SARS-CoV-2.

16. (canceled)

17. (canceled)

18. (canceled)

19. The composition of claim 1, which further comprises one or more additional pharmaceutically active agents.

20. The composition of claim 19, wherein the one or more additional pharmaceutically active agents are an antiviral agent, an antibacterial agent, an antifungal agent, and/or an anti-inflammation agent.

21. The method of claim 9, which further comprises administering to the patient in need thereof an effective amount of one or more additional pharmaceutically active agents.

22. The method of claim 21, wherein the one or more additional active agents are an antiviral agent, an antibacterial agent, an antifungal agent, and/or an anti-inflammation agent.

23. The method of claim 11, which further comprises administering to the patient in need thereof an effective amount of one or more additional pharmaceutically active agents.

24. The method of claim 23, wherein the one or more additional active agents are an anticancer agent.

25. A method for treating a disease or disorder comprising administering to a patient in need thereof an effective amount of a composition of claim 1, wherein the disease or disorder is a bacterial infection caused by tuberculosis, anthrax, Pseudomonas aeruginosa or Staphylococcus aureus, a type 2 diabetes mellitus, nonalcoholic fatty liver disease, artery constriction, endometriosis, neuropathic pain, rheumatoid arthritis, sclerodermatous graft-versus-host disease, or a systemic sclerosis.

26. A method for targeting one of more signaling pathways or biological processes including, but not limited to, uncoupling of oxidative phosphorylation, Wnt, mTORC1, STAT3, NF-κB, Notch, AKT/ERK/Src, AR-V7, C-Fos, C-Jun, E2F1, c-Myc, mGluRs, metabolic pathways, ROS, mitochondria, and pH comprising administering to a patient in need thereof an effective amount of a composition of claim 1.

27. The composition of claim 8, wherein OHPP and niclosamide are incorporated using a method selected from spray drying, spray chilling, extrusion, hot-melt extrusion, homogenization, milling such as jet milling, cryo-milling, ball milling, and colloid milling, vacuum drying, drum drying, film-forming, surface coating, printing, 3D printing, multi-layer forming, high-intensity blending, co-crystallization, personalized manufacturing of dosage forms, and a combination of different procedures.

28. The method of claim 9, wherein the oral bioavailability of niclosamide in the composition is at least 2 times higher than that of niclosamide alone as a pharmaceutical composition.

29. The method of claim 28, wherein the composition further comprises one or more absorption enhancers for further improved oral bioavailability of niclosamide.

30. The method of claim 9, wherein the highly branched glucan in the composition is octenylsuccinate hydroxypropyl phytoglycogen (OHPP).

31. The method of claim 11, wherein the patient is an animal.

32. The method of claim 11, wherein the patient is a human.

33. The composition of claim 19, wherein the oral bioavailability of niclosamide in the composition is at least 2 times higher than that of niclosamide alone as a pharmaceutical composition.

34. The composition of claim 33, wherein the composition further comprises one or more absorption enhancers for further improved oral bioavailability of niclosamide.

35. The composition of claim 19, wherein the highly branched glucan in the composition is octenylsuccinate hydroxypropyl phytoglycogen (OHPP).

36. The method of claim 25, wherein the oral bioavailability of niclosamide in the composition is at least 2 times higher than that of niclosamide alone as a pharmaceutical composition.

37. The method of claim 36, wherein the composition further comprises one or more absorption enhancers for further improved oral bioavailability of niclosamide.

38. The method of claim 25, wherein the highly branched glucan in the composition is octenylsuccinate hydroxypropyl phytoglycogen (OHPP).

39. The method of claim 26, wherein the oral bioavailability of niclosamide in the composition is at least 2 times higher than that of niclosamide alone as a pharmaceutical composition.

40. The method of claim 39, wherein the composition further comprises one or more absorption enhancers for further improved oral bioavailability of niclosamide.

41. The method of claim 26, wherein the highly branched glucan in the composition is octenylsuccinate hydroxypropyl phytoglycogen (OHPP).
